(19) United States
(12) Patent Application Publication
(10) Pub. No.: US 2016/0066882 A1
(43) Pub. Date: Mar. 10, 2016

(54) Intracardiac Ultrasound Imaging Delivery Catheter

(71) Applicant: Tyco Electronics Corporation, Berwyn, PA (US)

(72) Inventors: Alan D. Eskuri, Hanover, MN (US); Jay Pasquantonio, Damascus, OR (US)

(73) Assignee: Tyco Electronics Corporation, Berwyn, PA (US)

(21) Appl. No.: 14/483,066

(22) Filed: Sep. 10, 2014

(51) Int. Cl. A61B 8/12; A61B 8/08

(52) U.S. Cl. CPC A61B 8/12; A61B 8/0883

(57) ABSTRACT

An imaging catheter includes a delivery lumen and an imaging array. The imaging catheter is sized to be inserted within an introducer sheath. The delivery lumen facilitates insertion of a therapeutic device. An imager is arranged on an outside surface of a distal end of the imaging catheter. The imager collapses the distal end of the imaging catheter when the imager is within the introducer sheath. The distal end of the imaging catheter is allowed to expand when the imager exits the introducer sheath to facilitate delivery of the therapeutic device to a therapy site.

### BACKGROUND

The present invention relates generally to imaging catheters. More specifically, the present invention relates to an intracardiac ultrasound imaging catheter with a delivery lumen.

Imaging catheters are utilized to deliver an imager to a therapy site within a patient. For example, an imaging catheter may be utilized to place an imager in the atrium of the heart of the patient. The imager allows a doctor to observe the therapy site while positioning a therapeutic device at the therapy site to treat the patient.

Delivery of the imager begins by inserting an introducer sheath into the body of the patient to gain access to a vessel of the patient. The imaging catheter with an imager fitted at a distal end is inserted into the introducer sheath and fed through the vessel until reaching the point of therapy.

Typical imagers that may be fitted at the distal end of the catheter are so-called side-looking arrays, which do not have device delivery lumens. Side-looking arrays are delivered separately from the therapeutic device. That is, the therapeutic device is fed with a separate delivery catheter requiring a vascular access puncture and introducer sheath. Forward-looking ring arrays are an alternative in that the imager is arranged around the outside surface of the distal end of the imaging catheter. This facilitates delivery of the therapeutic device through a lumen defined in the imaging catheter. However, the increased diameter of the ring arrays increases the size of the required vascular access puncture and introducer sheath. In general, the level of discomfort experienced by the patient due to the insertion of the introducer sheath will increase with the diameter of the introducer sheath, as will the patient's recovery time.

### SUMMARY

In one aspect, an imaging catheter for insertion through an introducer sheath includes a delivery lumen that facilitates insertion of a therapeutic device. An imager is arranged on an outside surface of a distal end of the imaging catheter. The imager collapses the distal end of the imaging catheter when the imager is within the introducer sheath. The distal end of the imaging catheter is allowed to expand when the imager exits the introducer sheath to facilitate delivery of the therapeutic device to a therapy site.

### BRIEF DESCRIPTION OF THE DRAWINGS

FIG. 1A illustrates a cross-sectional side view of an exemplary imaging catheter retracted within an introducer sheath;

FIG. 1B illustrates a front view of the exemplary imaging catheter retracted within the introducer sheath;

FIG. 2A illustrates a cross-sectional side view of the exemplary imaging catheter after exiting the introducer sheath;

FIG. 2B illustrates a front view of the exemplary imaging catheter after exiting the introducer sheath;

FIGS. 3A and 3B illustrate cross-sectional side views of a therapeutic device moving through the exemplary imaging catheter;

FIGS. 4A and 4B illustrate cross-sectional side views of the imaging catheter contained within a containment sheath.

### DETAILED DESCRIPTION

An imaging catheter that overcomes the problems above is disclosed in detail below. Generally, the imaging catheter includes a collapsible distal end that allows an imager at the end of the imaging catheter to collapse into the lumen of the imaging catheter. This facilitates feeding the imaging catheter through an introducer sheath with a diameter smaller than that which would otherwise be required. The distal end of the imaging catheter is configured to expand or to be expanded after the distal end exits the introducer sheath, thus facilitating feeding a therapeutic device via the lumen of the imaging catheter to a therapy site. The reduction in the diameter of the introducer sheath results in less patient discomfort and a quicker recovery time for the patient.

FIG. 1A illustrates a cross-sectional side view of an exemplary imaging catheter retracted within an introducer sheath. Shown are the introducer sheath 100, an imaging catheter 105, and an imager 110. The introducer sheath 100 may have an inner diameter of between about 4 Fr and 30 Fr, and an outer diameter of between about 5 Fr and 35 Fr. The introducer sheath 100 may be formed from polyether block amide (PEBA), polyurethane, polyethylene, nylon, polyester, or other material suitable for insertion into the human body and flexible enough to be fed to a therapy site.

The portion of the imaging catheter 105 spaced apart from the distal end 120 may have an inner diameter compatible with the introducer sheath. The imaging catheter 105 may be formed from polyether block amide (PEBA), polyurethane, polyethylene, nylon, polyester, or other material suitable for insertion into the human body and flexible enough to be fed to a therapy site.

The distal end 120 of the imaging catheter 105 may comprise the same material properties as the rest of the imaging catheter 105, or different material properties. For example, the material for the distal end 120 may be selected to have a resiliency that is lower than the resiliency of the rest of the imaging catheter 105. Additionally or alternatively, the thickness of the imaging catheter 105 may be reduced at the distal end 120 or certain sections 112 of the distal end 120 to lower the resiliency of the distal end of the imaging catheter 105 and, therefore, allow the distal end of the imaging catheter 105 to collapse as illustrated.

The imager 110 may correspond to a forward-looking 2D array of transducers. Such an imager 110 produces an image that is clearer than an image produced by a forward-looking ring array imager because the ring array imager is open in the center, which causes the image quality to suffer. While a generally rectangular imager 110 is illustrated in the figures, the shape of the imager 110 may be changed to suit a given situation. For example, the imager 110 may have an octagonal shape. Other shapes are possible.

The transducers of the imager 110 may correspond to capacitive micro machined ultrasonic transducers (CMUTs), piezoelectric micro machined ultrasonic transducers (PMUTs), or a different type of transducer. The imager 110 may be positioned at the distal end 120 of the imaging catheter 105. In some implementations, the imager 110 is disposed within a housing material 117. The housing material 117 may be formed from polyether block amide (PEBA), polyurethane, polyethylene, nylon, polyester, or other material suitable for insertion into the human body. The housing material 117 may be selected to have a resiliency that is greater than the resiliency of the distal end 120 of the imaging catheter 105.

A group of conductors 115 for carrying imager 110 related signals may extend from the imager 110 and may be connected at an opposite end to imaging equipment (not shown). The conductors 115 may run along the outside surface of the imaging catheter in various configurations. For example, the conductors 115 may spiral around the outside surface of the imaging catheter to provide a desired turns/inch ratio. The conductors 115 may run in a generally straight direction along the outside surface. Other configurations are possible. In some implementations, the conductors 115 may be embedded within the sidewall of the imaging catheter 105, as illustrated in FIG. 1A. For example, the conductors 115 may be embedded within the imaging catheter 105 during an extrusion process for forming the imaging catheter 105. Alternatively, a channel (not shown) for feeding the conductors may be formed in the imaging catheter 105, and the conductors 115 may be fed through the channel in subsequent operations.

During operation, the imaging catheter 105 is inserted into the introducer sheath 100. Prior to insertion, an operator may pinch/squeeze the distal end of the imaging catheter 105 and imager 110 to collapse the imager 110 into the distal end of the imaging catheter 105, as illustrated in FIGS. 1A and 1B, to facilitate insertion of the imaging catheter 105 into the introducer sheath 100. For example, as illustrated in FIG. 1B, a sidewall portion of the distal end 120 of the imaging catheter 105 may fold inwards towards an opposite sidewall portion of the imaging catheter 105, thus closing or substantially closing the opening at the distal end of the imaging catheter 105. In some implementations, the operator may be required to pinch/squeeze the respective members until the distal end of the imaging catheter 105 is inserted into the introducer sheath 100. In alternative implementations, the distal end 120 of the imaging catheter 105 may be configured so that the imager 110 remains in the collapsed configuration without assistance.

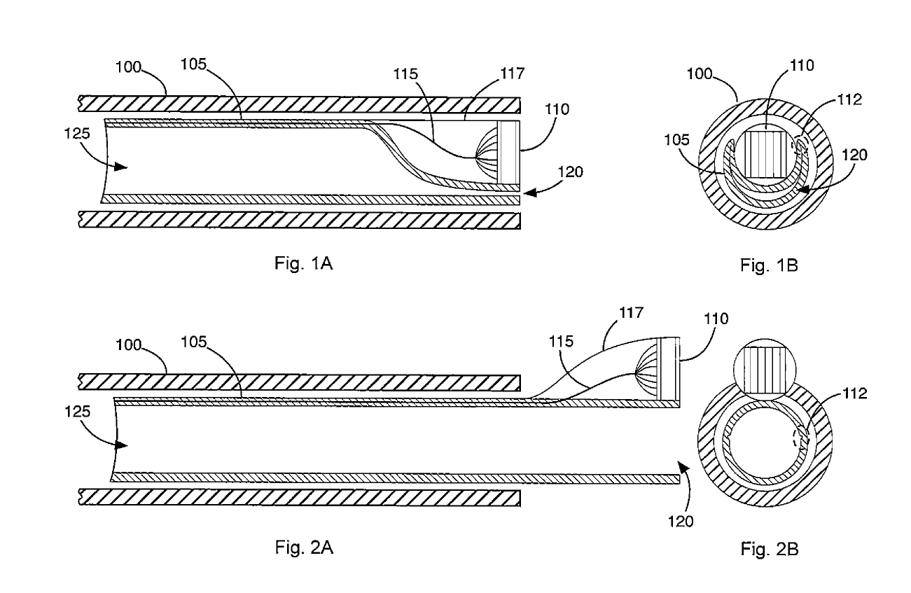

As illustrated in FIGS. 2A and 2B, when the distal end 120 of the imaging catheter 105 exits the introducer sheath 100, the resiliency of the distal end 120 of the imaging catheter 105 causes the distal end 120 of the imaging catheter 105 to open or at least open sufficiently enough to allow for a therapeutic device to be delivered via the lumen 125 of the imaging catheter 105.

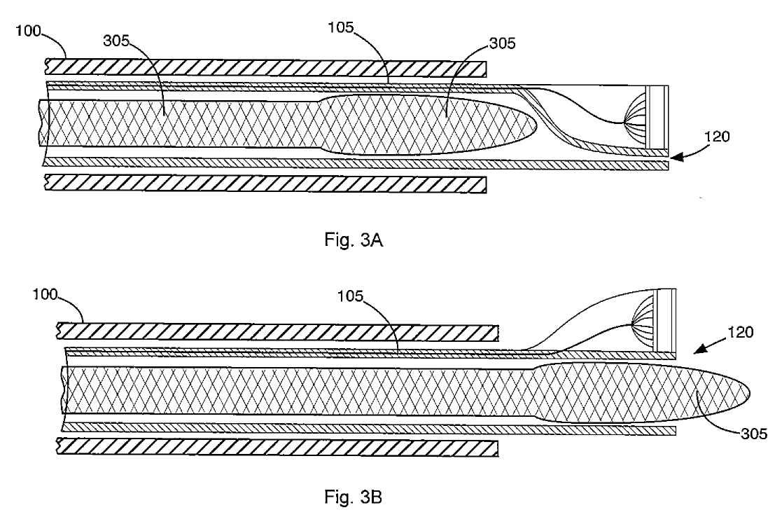

Referring to FIGS. 3A and 3B, in some implementations, the distal end 120 of the imaging catheter 105 may be configured so that movement of the therapeutic device 305 through the distal end 120 of the imaging catheter 105 is required to cause the distal end 120 to open. That is, the distal end 120 of the imaging catheter 105 may remain in the collapsed configuration of FIG. 1B and is pushed open as the therapeutic device 305 moves through the distal end 120 of the imaging catheter 105.

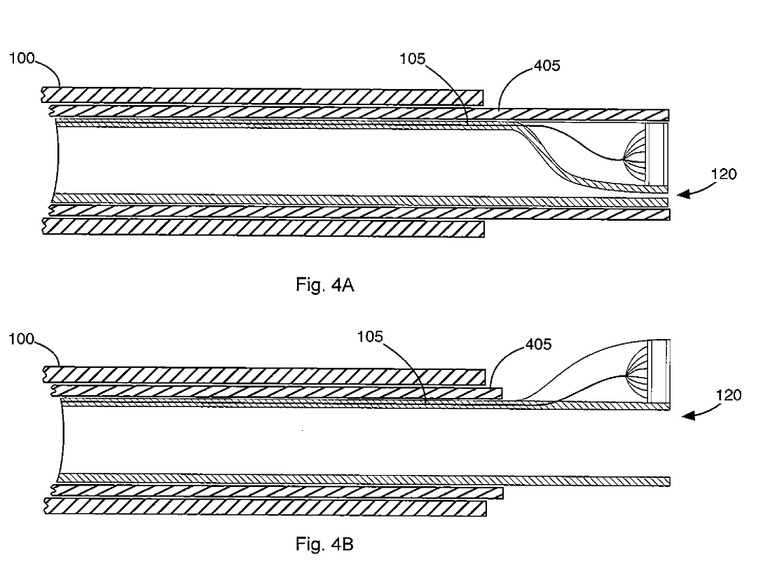

Referring to FIGS. 4A and 4B, in yet other implementations, a containment sheath 405 may be provided around the imaging catheter 105 to maintain the distal end 120 of the imaging catheter 105 in the collapsed configuration until the therapy site is reached. (See FIG. 4A.) After reaching the therapy site, the containment sheath 405 may be pulled back and the resiliency of the distal end 120 of the imaging catheter 105 may cause the distal end 120 to open, as illustrated in FIG. 4B.

As described above and illustrated in the figures, the imaging catheter 105 overcomes the problems associated with existing imaging catheter systems by providing a single catheter that facilitates both delivery of an imager and delivery of a therapeutic device. The reduction in the diameter of the imaging catheter 105 and number of vascular access sites required results in less patient discomfort and a quicker recovery time for the patient.

While the imaging catheter 105 has been described with reference to certain embodiments, it will be understood by those skilled in the art that various changes may be made and equivalents may be substituted without departing from the spirit and scope of the claims of the application. Various modifications may be made to adapt a particular situation or material to the teachings disclosed above without departing from the scope of the claims. Therefore, the claims should not be construed as being limited to any one of the particular embodiments disclosed, but to any embodiments that fall within the scope of the claims.

What is claimed is:

1. An imaging catheter for insertion through a lumen of an introducer sheath, the imaging catheter comprising:
a delivery lumen that facilitates insertion of a therapeutic device; and
an imager arranged on an outside surface of a distal end of the imaging catheter, wherein the imager collapses the distal end of the imaging catheter when the imager is within the introducer sheath, and wherein the distal end of the imaging catheter is allowed to expand when the imager exits the introducer sheath to facilitate delivery of the therapeutic device to a therapy site.

2. The imaging catheter of claim 1, wherein the imager is an ultrasonic imaging device.

3. The imaging catheter of claim 1, wherein the imager comprises a forward-looking 2D array of transducers.

4. The imaging catheter of claim 3, wherein the transducers are capacitive micro machined ultrasonic transducers (CMUTs) or piezoelectric micro machined ultrasonic transducers (PMUTs).

5. The imaging catheter of claim 1, wherein at least the distal end of the imaging catheter comprises a resilient material.

6. The imaging catheter of claim 5, wherein the material is selected from one of: polyether block amide (PEBA), polyurethane, polyethylene, nylon, and polyester.

7. The imaging catheter of claim 5, wherein the imager is arranged within a housing formed of a material less resilient than the imaging catheter.

8. The imaging catheter of claim 7, wherein the housing is bonded to an outside surface of the distal end of the imaging catheter.

9. The imaging catheter of claim 7, wherein the housing is formed integrally with the imaging catheter.

10. The imaging catheter of claim 1, further comprising a plurality of conductors coupled to the imager that extend to a proximal end of the imaging catheter, wherein the conductors are arranged on an outside surface of the imaging catheter.

11. The imaging catheter of claim 1, further comprising a plurality of conductors coupled to the imager that extend to a proximal end of the imaging catheter, wherein the conductors are embedded within a sidewall of the imaging catheter.

12. The imaging catheter of claim 1, wherein the distal end of the imaging catheter expands automatically when the imager exits the lumen of the introducer sheath.

13. The imaging catheter of claim 1, wherein the distal end of the imaging catheter remains in the collapsed configuration after the imager exits the lumen of the introducer sheath and is pushed open when the therapeutic device moves through the distal end of the imaging catheter.

14. The imaging catheter of claim 1, further comprising:
a containment sheath between the introducer sheath and the imaging catheter, wherein the imager collapses the distal end of the imaging catheter when the imager is within the containment sheath, and wherein the distal end of the imaging catheter is allowed to expand when the containment sheath is pulled in a direction so as to not cover the distal end of the imaging catheter.
